(19) United States
(12) Patent Application Publication
(10) Pub. No.: US 2013/0066062 A1
(43) Pub. Date: Mar. 14, 2013

(54) NUCLEIC ACID EXTRACTION METHOD, NUCLEIC ACID EXTRACTION REAGENT KIT, AND NUCLEIC ACID EXTRACTION REAGENT

(71) Applicants: Takuma Sano, Kanagawa (JP); Tomoyuki Chimuro, Kanagawa (JP)

(72) Inventors: Takuma Sano, Kanagawa (JP); Tomoyuki Chimuro, Kanagawa (JP)

(73) Assignee: Kanto Kagaku Kabushiki Kaisha, Tokyo (JP)

(21) Appl. No.: 13/592,390

(22) Filed: Aug. 23, 2012

(30) Foreign Application Priority Data
Aug. 26, 2011 (JP) 2011-185275

(51) Int. Cl. C07H 1/08; C09K 3/00

(52) U.S. Cl. 536/25.41; 252/182.11

(57) ABSTRACT

A method is provided that is suitable for various test samples and that prepares nucleic acid templates directly usable for gene amplification reaction such as PCR method or RT-PCR method conveniently and promptly, preferably under a mild condition. A nucleic acid extraction method is provided comprising a process of contacting a nucleic acid extraction reagent with a test sample, wherein the nucleic acid extraction reagent comprises zwitterionic buffer solution. Preferably, the nucleic acid extraction reagent comprises a surfactant and a proteolytic enzyme.

### FIELD OF THE INVENTION

The present invention relates to a nucleic acid extraction method, a nucleic acid extraction reagent kit, and a nucleic acid extraction reagent, for simply and quickly preparing nucleic acids (DNA or RNA) from a test sample, wherein the nucleic acids (DNA or RNA) are directly applicable for gene amplification reaction such as PCR method or RT-PCR method.

### BACKGROUND OF THE INVENTION

Generally, nucleic acids supplied for gene amplification reaction are prepared after disrupting cell walls or cell membranes of a test sample physically or chemically, then denaturing proteins of the test sample, and only nucleic acids being isolated. In this case, strong alkaline sodium hydroxide solution, organic solution such as phenol, chloroform, etc., or toxic 2-mercaptoethanol is used as a reagent. However, all of these reagents require careful handling. In addition, if such reagent remains with nucleic acids after preparation, it is possible to adversely affect subsequent gene amplification reaction. To circumvent this problem, further operations for nucleic acid purification with a use of centrifuge separation, column chromatography, etc., or adding extra reagents that have effect in suppressing interference reaction into reaction solutions in order to carry out gene amplification reaction even with the presence of some contaminants, are performed frequently.

Of those mentioned above, the former purification operation commonly employ a method comprising the steps of: preparing a solution (adsorption solution) by adding a sample comprising nucleic acids to a dissolving solution comprising a buffer solution generally used in biochemical experiments; contacting the solution with particles and/or substrates made of silica, glass, latex, polystyrene, cellulose, agarose, or basic protein (protamine, etc.), or solid materials in which ionic or hydrophobic functional groups are bonded to surfaces of the particles and/or substrates; thereafter contacting a collectate with the solid materials and then collecting eluate comprising nucleic acids. For example, Patent Document 2 discloses a method characterized by centrifuging solid materials for collection of adsorption solution or collectate, while Patent Document 3 discloses a method of using zwitterionic buffer solution in adsorption solution and collectate. Moreover, a method based on the molecular weight difference between nucleic acids and contaminants (Gel Filtration Chromatography, or Size Exclusion Chromatography, etc.) is also commonly employed.

Researches on the latter addition of reagents are also frequently conducted. For example, Patent Document 1 discloses a method of adding/mixing zwitterionic buffers with nucleic acid extract in a gene amplification reaction in order to suppress the interference of the gene amplification reaction caused by contaminants which are derived from a test sample.

However, depending on the origin of test samples, some test samples may contain a high percentage of the components that interfere the above-mentioned nucleic acid extraction/purification operation. In this case, it is usually necessary to further employ purification methods that are specific to each test sample.

On the other hand, domestic and foreign reagent manufacturers currently develop various nucleic acid extraction/purification kits and sell them as products for improving convenience of users. However, for most of the kits, there is a limitation that preparable nucleic acids are limited to either DNA or RNA and test samples are limited individually. Thus, users have to select the most suitable method and kit from various options, considering types of test samples, their conditions, and objects to be assayed such as the types of nucleic acids, etc.

When test samples are sufficiently available, when contaminants contained in test samples are presumable, or when the DNA or RNA as objects to be assayed are determined, it allows selecting the most suitable method and a combination of methods from the existing methods. However, when test samples are only available in a small amount, when there is lack of information on contaminants contained in test samples, or when a plurality of DNA or RNA as objects to be assayed are present, it may be difficult to conduct preparatory investigation on the suitability with existing methods. Therefore, a nucleic acid extraction method applicable for a wide range even in such case, e.g., without a preparatory investigation, is desired.

Moreover, when the number of test sample is significantly large, as it is needed to prepare samples applicable for PCR or RT-PCR determination in a short time, it would be hard to employ a complicated method that needs purification operations before and after nucleic acid extraction. Thus, a nucleic acid extraction method without a purification operation is desired.

For the purpose of responding to such needs, reagent kits, which can extract nucleic acids that are directly applicable for gene amplification reaction conveniently in a short time (e.g., Product Name: CellEase (manufactured by Biocosm Inc.), Product Name: Cica Geneus DNA Extraction Reagent (manufactured by Kanto Chemical Co., Inc.)), are on sale. However, as the enclosed documents of these reagent kits (e.g., Non-Patent Document 1) only describe nucleic acids DNA, it is unclear whether it is applicable for RNA, and further, there is no any disclosure about their compositions.

Moreover, Patent Documents 4 and 5 disclose a method, with a use of composition only composed of reagents that are not toxic to the human body, for extracting/preparing nucleic acids that are directly applicable for gene amplification reaction without any purification operation such as Column Chromatography, etc. Examples of Patent Document 4 describe a result of extracting DNA from Cryptosporidium and DNA from Legionella (a gram-negative bacteria) and HeLa cells (human cultured cells) which are regarded as representative examples of test samples without cell walls. Further, Examples of Patent Document 5 additionally describe a result of extracting DNA from rice seeds (plant) with cell wall disruption through boiling treatment. However, it is unclear whether these methods are applicable for other test samples such as gram-positive bacterium or fungus/yeasts, etc., the demand for which is growing.

Furthermore, Patent Documents 4 and 5 specifically shows that nucleic acids DNA can be extracted, but there is no specific description whether RNA extraction and subsequent analysis can be conducted. Moreover, the methods described in these documents have many steps with repeated operation of mixing reagents and heating, which is not convenient and may damage the extracted nucleic acids. Therefore, a method for preparing less damaged nucleic acids under a milder condition conveniently in a short time is desired.

As a method for extracting RNA, guanidine-cesium chloride ultracentrifugation method is known, wherein RNA degrading enzymes are inactivated by guanidine thiocyanate and RNA is collected by density gradient centrifugation (e.g., Non-Patent Document 2). This method is advantageous to collect high-purity RNA, but it has problems that an ultracentrifuge is needed, which takes a long time (e.g., several days), and small molecular RNA cannot be collected.

Moreover, APGC (Acid Guanidinium-Phenol-Chloroform) is known, wherein RNA degrading enzymes are inactivated by guanidinium thiocyanate and the supernatant (supernatant contains RNA, middle layer contains proteins, and bottom layer contains DNA) is collected after centrifugation with addition of phenol/chloroform (e.g., Non-Patent Document 3). This method is currently employed broadly with advantages of using a conventional centrifuge rather than an ultracentrifuge and costing less, but it has problems that it needs to use toxic phenol and chloroform and method operation takes a relatively long time (e.g., several hours).

Furthermore, a method of using silica membranes (SpinColumns) is known, wherein nucleic acids are purified by removing liquid, which is done by passing samples that contain nucleic acid-binding solid phase through silica membrane, and subsequently isolating the nucleic acid from solid phase (e.g., Non-Patent Document 2). This method has an advantage of collecting high-purity nucleic acids by a relative simple operation, but it has problems that it needs to conduct centrifugation repeatedly for collecting highly purified nucleic acids, it takes a relatively long time (30 minutes to several hours) for conducting this purification operation, and it needs to consider the compatibility of extracting solutions and silica membranes depending on the type of samples (to select solutions that would not isolate nucleic acids from solid phase).

Moreover, a method of using magnetic beads is known, wherein nucleic acids (DNA and RNA) reversibly bind to magnetic beads, after collecting beads by magnets, nucleic acids are purified by isolating them from the beads (e.g., Non-Patent Document 4). Such method has an advantage of collecting high-purified nucleic acids by convenient operation process compared with centrifugation operation, but it needs to consider the compatibility of extracting solutions and magnetic beads depending on the type of samples (to select solutions that would not isolate nucleic acids from magnetic beads) and it highly costs.

As mentioned above, the RNA extraction methods conducted so far have to go through many processes such as mixing reagents, pipetting, and centrifugation, etc., and need time for preparation. Thus, RNA with low stability is degraded easily in the process of preparation. Specifically, when the amount of RNA contained in a sample is small, there is a problem that extraction cannot be carried out. Moreover, when there are many extraction processes, there is a problem that Ribonuclease generally present in the environment may be contained through equipments or operators.

Therefore, methods for preparing RNA with few processes in a short time, specifically, methods for preparing RNA templates usable for gene amplification reaction, are desired.

### CITATION LIST

### Patent Document

[Patent Document 1] JP 2008-531039 A
[Patent Document 2] JP H02-289596 A
[Patent Document 3] JP 2009-284784 A
[Patent Document 4] JP 2006-061041 A
[Patent Document 5] WO 2007-116450 A1

### Non-Patent Document

[Non-Patent Document 1] “Cica Geneus DNA Extraction Reagent”, enclosed document.
[Non-Patent Document 2] Chirgwin, J. M. et al. (1979) Isolation of biologically active ribonucleic acid from sources enriched in ribonuclease. Biochemistry 18 (24): 5294-5299
[Non-Patent Document 3] Chomczynski, P. and Sacchi, N. (1987) Single-step method of RNA isolation by acid guanidinium thiocyanate-phenol-chloroform extraction. Analytical Biochemistry 162 (1): 156-159
[Non-Patent Document 4] DeAngelis, M. M. et al. (1995) Solid-phase reversible immobilization for the isolation of PCR products. Nucleic Acids Research 23 (22): 4742-4743

### SUMMARY OF THE INVENTION

### Technical Problem

Accordingly, problems to be solved by the present invention is to provide a method that is suitable for various test samples and that prepares nucleic acid templates directly usable for gene amplification reaction such as PCR method or RT-PCR method conveniently and promptly, preferably under a mild condition.

### Solution to Problems

While the present inventors have been carrying out an intensive investigation in view of the above-mentioned problems, it has been found that a usage of nucleic acid extraction reagent containing zwitterionic buffer solution can suppress the interference of gene amplification reaction caused by the contaminants extracted with DNA and RNA, and the present invention has thus been accomplished.

That is, the present invention relates to following inventions.

(1) A nucleic acid extraction method comprising a process of contacting a nucleic acid extraction reagent with a test sample, wherein the nucleic acid extraction reagent comprises a zwitterionic buffer solution.

(2) The nucleic acid extraction method according to (1), wherein the nucleic acid extraction reagent comprises at least one protease and/or at least one surfactant.

(3) The nucleic acid extraction method according to (2), wherein the protease comprises Protease K.

(4) The nucleic acid extraction method according to (2) or (3), wherein concentration of the protease in the nucleic acid extraction reagent is 0.09 to 45 U/mL.

(5) The nucleic acid extraction method according to any one of (2) to (4), wherein the surfactant comprises that having a steroid backbone.

(6) The nucleic acid extraction method according to any one of (2) to (5), wherein the surfactant comprises glycocholic acid or its salt.

(7) The nucleic acid extraction method according to any one of (2) to (6), wherein concentration of the surfactants in the nucleic acid extraction reagent is 0.009 to 9 mmol/L.

(8) The nucleic acid extraction method according to any one of (1) to (7), wherein the zwitterionic buffer solution is Good's buffer.

(9) The nucleic acid extraction method according to any one of (1) to (8), wherein the zwitterionic buffer solution comprises tricine.

(10) The nucleic acid extraction method according to any one of (1) to (9), wherein concentration of the buffer in the nucleic acid extraction reagent is 9 to 364 mmol/L.

(11) The nucleic acid extraction method according to any one of (1) to (10), wherein the test sample is a sample derived from organisms having cell walls.

(12) The nucleic acid extraction method according to any one of (1) to (11), wherein the organisms having cell walls comprises Gram-positive bacterium, fungus, or yeasts.

(13) The nucleic acid extraction method according to any one of (1) to (12), wherein DNA and/or RNA are/is extracted as nucleic acids from the test samples.

(14) The nucleic acid extraction method according to any one of (1) to (13), wherein the nucleic acid extraction reagent that is contacted with the test sample and extract nucleic acids from the test sample is used for nucleic acid amplification reaction.

(15) A nucleic acid extraction reagent comprising a zwitterionic buffer solution.

(16) A nucleic acid extraction reagent kit comprising a zwitterionic buffer solution and each component that compose a nucleic acid extraction reagent.

### Advantageous Effects of the Invention

According to the present invention, it is not needed to consider types of test samples or whether there is any contaminant, and nucleic acids can be extracted conveniently and promptly from a very small amount of test samples through a certain operation. Furthermore, templates used for PCR and RT-PCR can be prepared. Moreover, while the demand for genetic test is increasing currently in the testing market, such as tests for food poisoning or infectious diseases, a use of the present invention allows significantly shortening the operation of sample preparation (preparing templates for PCR and RT-PCR) and saving labor, which has been a bottleneck so far.

Specifically, since RNA is easily degraded during the extraction and purification operations, it is difficult to be handled by conventional techniques. However, such RNA can be suitably extracted according to the present invention, because purification operation can be omitted and RNA damage and degradation in the purification operation are prevented. Furthermore, RNA prepared by the present invention is different from that collected by conventional techniques for its excellent storage stability, which allows collecting reliable data even when a further measurement is needed.

Furthermore, the present invention can prevent the bad effect caused by contaminants in gene amplification reaction beforehand at the step of nucleic acid extraction. Thus, it allows using buffer solutions that are suitable for gene amplification reaction purposes in gene amplification reaction, and is not limited by a specific buffer solution in gene amplification reaction like the method described in Patent Document 1.

Moreover, specifically, when a nucleic acid extraction reagent contains a combination of surfactants and proteases, specifically a combination of glycocholic acid and Protease K, nucleic acids can be extracted suitably even when test sample is cells having cell walls. Thus, the present invention is applicable for more extensive test examples.

### BRIEF DESCRIPTION OF THE DRAWINGS

FIG. 1 is a flowchart showing one of the preferable embodiments of the nucleic acid extraction method of the present invention.

FIG. 2 is a figure observed after agarose electrophoresis for amplified DNA that are extracted from a suspension of the budding yeast by Reagent 1 on a stepwise change in concentration of Protease K.

FIG. 3 is a figure observed after agarose electrophoresis for amplified DNA that are extracted from a suspension of the budding yeast by Reagent 1 on a stepwise change in concentration of sodium glycocholate.

FIG. 4 is a figure observed after agarose electrophoresis for amplified DNA that are extracted from a suspension of the budding yeast containing human blood component by Reagent 1 on a stepwise change in concentration range of tricine.

FIG. 5 is an observation figure showing a result after agarose electrophoresis for comparing DNA content extracted from each suspension of Staphylococcus aureus, Escherichia coli O157 and budding yeast, by the nucleic acid extraction method of the present invention and hot water extraction method respectively.

FIG. 6 is an observation figure showing a result after agarose electrophoresis for comparing DNA content extracted from each suspension of Staphylococcus aureus, Escherichia coli O157 and budding yeast, by an extraction method using a conventional reagent and hot water extraction method respectively.

FIG. 7 is an observation figure showing a result after agarose electrophoresis for comparing DNA content extracted from animal cells, by using a nucleic acid extraction reagent kit of the present invention and an existing product (CellEase Mouse Tail) respectively.

FIG. 8 is an observation figure showing a result after agarose electrophoresis for comparing RNA content extracted from each suspension of Staphylococcus aureus, Escherichia coli O157 and budding yeast, by the nucleic acid extraction method of the present invention and hot water extraction method respectively.

FIG. 9 is an observation figure of agarose electrophoresis showing a result of DNA extraction from a suspension of the budding yeast mixed with blood component by using the method of the present invention.

FIG. 10 is an observation figure of agarose electrophoresis showing a result of RNA extraction from a suspension of the budding yeast mixed with blood component by using the method of the present invention.

### DETAILED DESCRIPTION OF THE INVENTION

The present invention is explained in detail below based on the suitable embodiments of the present invention.

Before explaining the nucleic acid extraction method of the present invention, the nucleic acid extraction reagent and nucleic acid extraction reagent kit of the present invention will be explained first.

The nucleic acid extraction reagent of the present invention is used for extracting nucleic acids, specifically DNA and/or RNA, from test samples. Further, the nucleic acid extraction reagent of the present invention, for example, is a reagent for extracting nucleic acids used for gene amplification reaction after the nucleic acids are extracted from test samples.

Further, unless otherwise noted, the term “nucleic acid(s)” of the present invention encompasses naturally derived DNA (desoxyribonucleic acids) and RNA (ribonucleic acids), and derivatives thereof (methylated forms, oxidized forms or dimmers), artificially produced DNA and RNA mimics (primers or non-natural nucleic acid bases being comprised, etc.), and their modifiers (thiophosphoric acid esters, thiolated forms, phosphorylated forms, amidated forms, biotinylated forms and fluorescent markers).

Moreover, unless otherwise noted, the term “extraction” of the present invention refers to exporting materials (contents), which are stored in shells and are separated from the outside environment by biological membranes (cell walls, cell membranes, nuclear membranes or mitochondrial membranes), into a solution that breaks the shell structure by being contacted with the shells, on the basis of the affinity and solubility between the contents and the solution.

Moreover, the nucleic acid extraction reagent of the present invention comprises a zwitterionic buffer solution.

A zwitterionic buffer solution comprises at least one zwitterionic compound as buffer. Within a certain pH range, the compound is present in the zwitterionic buffer solution as zwitterions with both positive and negative charges at the same time. Including such zwitterionic buffer solution in a nucleic acid extraction reagent can decrease the effect that is caused by the contaminants contained in test samples during extraction and gene amplification reaction. As a result, nucleic acids can be extracted and purification operation after nucleic acid extraction can be omitted without considering the types of test samples and whether there are contaminants. The reason of obtaining such effect has not been clarified completely yet. However, for example, it can be regarded that a zwitterionic compound traps positive charged contaminants by its negative charges, wherein the positive charged contaminants are regarded as interfering nucleic acids binding to DNA polymerase by attaching nucleic acids with negative charges, and at the same time, the zwitterionic compound also traps negative charged contaminants by its positive charges, wherein the negative charged contaminants are regarded as inhibiting the activity of DNA polymerase by electrically neutralizing divalent cations (such as magnesium ion), which are needed for activating DNA polymerase, and that the zwitterionic compound can flocculate and precipitate the contaminants. As a result, the effects to nucleic acids and polymerase by the contaminants are decreased.

As a zwitterionic buffer solution contained in the nucleic acid extraction reagent, it is not particularly limited, for example, Good's buffer solution can be used. Specifically, buffer solutions can be those comprising, as buffers, one or more selected from 2-Morpholinoethanesulfonic acid (MES), N-(2-Acetamido)iminodiacetic acid (ADA), N-(2-Acetamido)-2-aminoethanesulfonic acid (ACES), N,N-Bis(2-hydroxyethyl)-2-aminoethanesulfonic acid (BES), N,N-Di(2-hydroxyethyl)glycine (Bicine), Bis(2-hydroxyethyl)iminotris(hydroxymethyl)methane (Bis-Tris), N-Cyclohexyl-3-aminopropanesulfonic acid (CAPS), N-Cyclohexyl-2-aminoethanesulfonic acid (CHES), 3-[4-(2-Hydroxyethyl)-1-piperazinyl]propanesulfonic acid (EPPS), 2-[4-(2-Hydroxyethyl)-1-piperazinyl]ethanesulfonic acid (HEPES), 2-Hydroxy-3-[4-(2-hydroxyethyl)-1-piperazinyl]propanesulfonic acid (HEPPSO), 3-Morpholinopropanesulfonic acid (MOPS), 2-Hydroxy-3-morpholinopropanesulfonic acid (MOPSO), piperazine-1,4-bis(2-ethanesulfonic acid) (PIPES), piperazine-1,4-bis(2-hydroxy-3-propanesulfonic acid) (POPSO), N-Tris(hydroxymethyl)methyl-3-aminopropanesulfonic acid (TAPS), 2-Hydroxy-N-tris(hydroxymethyl)methyl-3-aminopropanesulfonic acid (TAPSO), N-[Tris(hydroxymethyl)methyl]-2-ethanesulfonic acid (TES), N-[Tris(hydroxymethyl)methyl]glycine (Tricine), N,N,N-trimethyl-2-aminoethaneaminium chloride (Cholamine chloride), acetamidoglycine and glycinamide, and salts thereof as the zwitterionic buffer solution. Preferably, buffer solutions comprising, as buffers, one or more selected from N,N-Di(2-hydroxyethyl)glycine (Bicine), Bis(2-hydroxyethyl)iminotris(hydroxymethyl)methane (Bis-Tris), N-Tris(hydroxymethyl)methyl-3-aminopropanesulfonic acid (TAPS), 2-Hydroxy-N-tris(hydroxymethyl)methyl-3-aminopropanesulfonic acid (TAPSO), N-[Tris(hydroxymethyl)methyl]-2-ethanesulfonic acid (TES), N-[Tris(hydroxymethyl)methyl]glycine (Tricine) and 2-[4-(2-Hydroxyethyl)-1-piperazinyl]ethanesulfonic acid (HEPES) as buffers can be used. More preferably, buffer solutions containing one or more selected from among Bicine, Tricine and 2-[4-(2-Hydroxyethyl)-1-piperazinyl]ethanesulfonic acid can be used as the zwitterionic buffer solution. Further more preferably, buffer solutions comprising Tricine as a buffer can be used as the zwitterionic buffer solution.

Moreover, the zwitterionic buffer solution may comprise a buffering agent other than the above zwitterionic compounds. To make the activity of enzymes relating to nucleic acid extraction be optimal, such buffering agents can be those that modulate the condition from neutral to slightly alkaline. Preferably it can be, but not particularly limited to, trishydroxy-amino-methane, phosphoric acid, boric acid, 3,3-dimethylglutaric acid, maleic acid, imidazole, triethanolamine, diethanolamine, pyrophosphoric acid, glycine, or salts thereof (e.g., sodium salts, potassium salts, or hydrochloride salts). One or more of these buffering agents can be used in combination.

Concentration of the buffering agent in the nucleic acid extraction reagent is different according to the type of the buffering agent. Though the concentration of the buffering agent is not particularly limited, an excessive amount of the buffering agent compared with the amount of contaminants must be present in the zwitterionic buffer solution to completely neutralize cationic and/or anionic contaminants, and the concentration must be set in a degree that the buffering agent itself would not interfere gene amplification reaction. From the above viewpoints, preferably, the concentration of the buffering agent is 9 to 364 mmol/L. More preferably, it is 91 to 182 mmol/L. Specifically, when the concentration of zwitterionic compounds, preferably, the concentration of tricine, is within the above range, the effect of the present invention is exhibited more remarkably.

Moreover, preferably, the nucleic acid extraction reagent comprises at least one protease and/or at least one surfactant. Accordingly, cell walls, cell membranes and nuclear membranes etc. can be disrupted sufficiently and nucleic acids can be extracted more easily under a relatively mild condition. More preferably, the nucleic acid extraction reagent comprises at least one protease and at least one surfactant. Accordingly, through the coordinated effects of the protease and the surfactant, cell walls, cell membranes and nuclear membranes etc. can be disrupted more suitably.

Such protease is not particularly limited, if it has an ability to degrade proteins and disrupt at least one of cell walls, cell membranes and nuclear membranes (in the case of eukaryotes) in a degree that nucleic acids of test samples can be extracted, and, for example, one or more proteases selected from Protease K, trypsin, chymotrypsin, and subtilisin, etc. those belong to serine protease, pepsin and cathepsin D, etc. those belong to aspartic protease, and papain, cathepsin, caspase, and calpain etc. those belong to cysteine protease, can be used. Preferably, Protease K, which has broad substrate specificity of proteins and keeps activity even when chelating agents or denaturing agents are present, is used.

Moreover, while concentration of the protease in the nucleic acid extraction reagent depends on the type of the protease and is not particularly limited, for example, preferably, it is 0.09 to 45 U/ml, and more preferably, it is 0.9 to 45 U/ml. Due to this, during nucleic acid extraction, disruption of cell walls, cell membranes and nuclear membranes can be accelerated, and the nucleic acid extraction can be effectively carried out. And the amplified amount of nucleic acid sequence can be sufficient during gene amplification reaction. Specifically, when Protease K in the above-mentioned concentration is comprised in the nucleic acid extraction reagent as a protease, the above-mentioned effect can remarkably be obtained.

Furthermore, the surfactant is not particularly limited if it contributes to the disruption of at least one of cell walls, cell membranes and nuclear membranes. Preferably, a surfactant having the steroid backbone can be used, which specifically includes deoxycholic acid, glycocholic acid or salts thereof (e.g., sodium salts or potassium salts), and one or more of these can be used in combination. More preferably, glycocholic acid or salts thereof (e.g., sodium salts or potassium salts) is used.

Moreover, concentration of the surfactant in the nucleic acid extraction reagent is different according to the type of the surfactant. For example, when the surfactant is glycocholic acid, preferably, the concentration is 0.009 to 9 mmol/L, and more preferably, the concentration is 0.9 to 9 mmol/L. When the surfactant is deoxycholic acid, preferably, the concentration is 0.009 to 9 mmol/L, and more preferably, the concentration is 0.9 to 9 mmol/L. Due to this, during nucleic acid extraction, disruption of at least one of cell walls, cell membranes and nuclear membranes can be accelerated, and the nucleic acid extraction can be effectively carried out. And the amplified amount of nucleic acid sequence can be sufficient during gene amplification reaction. Specifically, when glycocholic acid or salts thereof in the above-mentioned concentration is contained in nucleic acid extraction reagent as a surfactant, the above-mentioned effect can remarkably be obtained.

Furthermore, the nucleic acid extraction reagent may comprise a component other than those mentioned above. Such component includes, but is not particularly limited to, e.g., a chelating agent such as ethylenediaminetetraacetic acid (EDTA), O,O′-Bis(2-aminophenyl)ethyleneglycol-tetraacetic acid (BAPTA), trans-1,2-Diaminocyclohexane-tetraacetic acid (CyDTA) or salts thereof, a calcium salt such as calcium acetate, calcium formate or calcium chloride, a non-specific adsorption inhibitor such as sodium chloride, potassium chloride or sodium sulfate, an antifreezing agent such as glycerin. One or more of these can be used in a combination with a range in which the enzyme reactions of proteases (e.g., Protease K) and deoxyribonuclease (e.g., DNase I), and the gene amplification reaction (e.g., DNA polymerase for PCR) will not be interfered.

Moreover, when the nucleic acid extraction reagent comprises a surfactant and a protease at the same time, preferably, it contains glycocholic acid as the surfactant and Protease K as the protease. Due to such combination, cell walls, cell membranes and nuclear membranes in test samples can be disrupted more easily and promptly, and nucleic acids can be extracted more effectively, under a sufficiently mild condition.

Furthermore, pH of the nucleic acid extraction reagent at 20° C. is not particularly limited, but the enzyme reactions of proteases (e.g., Protease K) and deoxyribonuclease (e.g., DNase I), and gene amplification reaction (e.g., DNA polymerase for PCR) should be kept active. Preferably, the pH is 7.5 to 8.7, and more preferably, the pH is 8.0 to 8.5.

The nucleic acid extraction reagent kit of the present invention at least includes zwitterionic buffer and each component that compose a nucleic acid extraction reagent as mentioned above.

As for the nucleic acid extraction reagent, each of the components may be present in a mixed state in the nucleic acid extraction reagent kit. From the viewpoints of improving the preservation stability and decreasing the manufacturing cost, these components may be present separately according to the need.

Moreover, the nucleic acid extraction reagent kit may include other reagents according to its purpose. For example, the nucleic acid extraction reagent kit can include one or a combination of more reagents selected from nucleolytic reagents for degrading nucleic acids other than those of interest (e.g., DNA-degrading reagents or RNA-degrading reagents), proteins that inhibit the activity of ribonuclease (RNase Inhibitor), gene amplification reagents for PCR, etc. (DNA polymerase, primers, nuclear acid bases, or buffer solutions containing magnesium), and electrophoresis related reagents for detecting gene amplification products (agarose gels, molecular weight markers, migration markers, or detecting reagents), etc and their components.

Furthermore, the nucleic acid extraction reagent kit of the present invention may include equipments for conducting nucleic acid extraction methods as described below (e.g., closed containers, etc.).

The nucleic acid extraction reagent and the nucleic acid extraction reagent kit of the present invention as described above can be suitably applied to the nucleic acid extraction method of the present invention as described below.

Next, the nucleic acid extraction method of the present invention is explained.

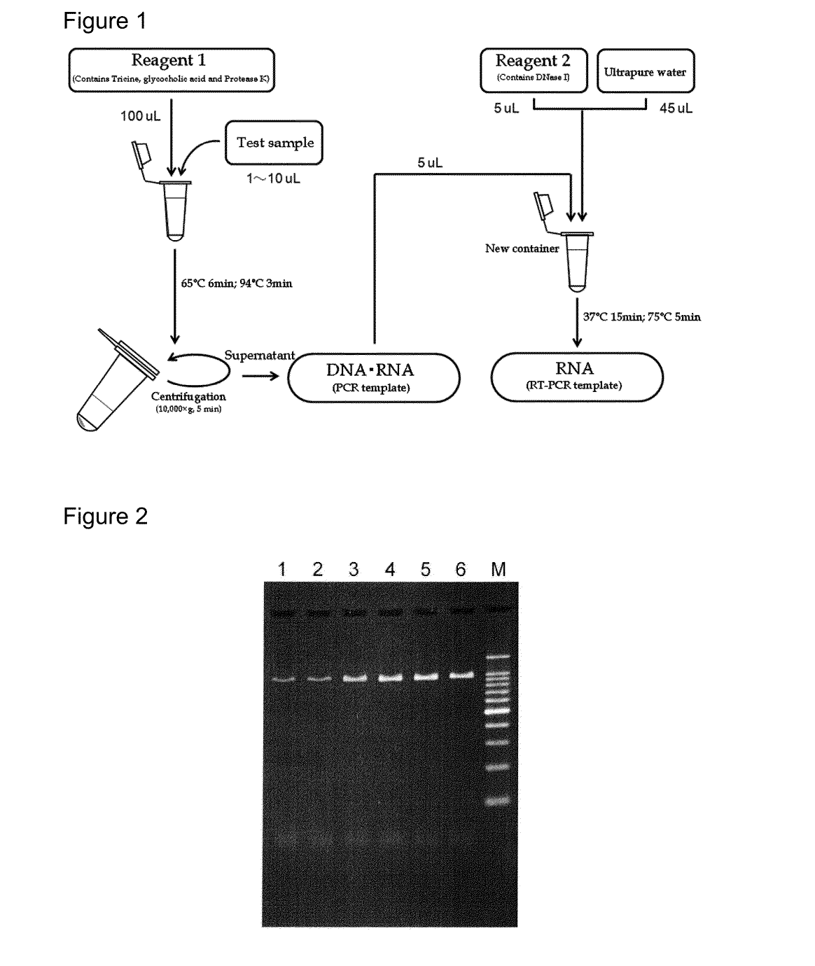

FIG. 1 is a flowchart showing one of the preferable embodiments of the nucleic acid extraction method of the present invention. Further, the components in the reagent or the reaction conditions described in the figure only indicate one example of the embodiments of the present invention, to which the present invention is not intended to be limited.

The nucleic acid extraction method of the present invention comprises the process of contacting nucleic acid extraction reagent that comprises zwitterionic buffer solutions (Reagent 1 in FIG. 1) with test samples.

In this embodiment, firstly, a mixture is obtained by mixing the test sample with the above-mentioned nucleic acid extraction reagent to contact the test sample and the nucleic acid extraction reagent.

In the nucleic acid extraction method of the present invention, cells that comprise nucleic acids (microorganisms, animals or plants) or virus are used as the test samples. Such test samples are not particularly limited, but preferably, the test samples are derived from organisms (prokaryotes or eukaryotes) having cell walls. More preferably, the test sample is derived from Gram-positive bacteria, fungus, or yeasts. Furthermore preferably, the test sample is derived from Staphylococcus aureus or budding yeasts.

The derivation of samples that contain these test samples is not particularly limited, and it may be anything. For example, as for samples that contain microorganisms and virus, it can be blood, urine, fecal material, mucus (e.g., the vagina, the cervix, the buccal cavity, or the cavum nasi, etc.), food, crops, water and sewage, natural water (e.g., river water, lake water, underground water, rain water, or sea water), or soil, etc. The primary sample collected by each suitable method can be used directly, or after culturing the primary sample.

Moreover, for example, as for samples that contain animal samples, it can be blood, urine, fecal matter, mucus (e.g., the vagina, the cervix, the buccal cavity, or the cavum nasi, etc.), skin, hair root, food (meat), or biopsy samples removed by surgery. The primary sample collected by each suitable method can be used directly, or after culturing the primary sample. Moreover, frozen cells of these samples or sections cut after paraffin embedding also can be used.

Furthermore, for example, as for samples derived from plants, it can be seeds, fruits, seed coats, tubes, leaves or roots.

Further, in the nucleic acid extraction method of the present invention, nucleic acids are suitably extractable even when contaminants are contained in the above-mentioned test samples. That is, for test samples obtained with the mixture of contaminants, the method of the present invention is applicable without examining the presence of contaminants. In the present specification, contaminants, not limited to molecular weight, are regarded as compositions comprising one or more of those selected from compounds with positive or negative charges, compounds that inhibit the activity of proteases (e.g., Protease K) and deoxyribonuclease (e.g., DNase I) and enzymes for gene amplification (e.g., DNA polymerase for PCR), and compounds that decrease the stability of these enzymes. Such contaminants can be, but are not particularly limited to, for example, serum, blood cells, urine, fecal material, mucus, spinal fluid, saliva, soil, cell debris, culture medium, proteins, lipids, fats, polysaccharides, oligosaccharides, dye, metallic salts, acid salts, basic salts, antibodies, drugs, or surfactants. Among what have been mentioned above, nucleic acid amplification reaction tends to be interfered when serum, urine, mucus, cell debris, culture medium, metallic salts, acid salts or basic salts are present in the extraction liquid. However, such interference can be inhibited by the present invention even when such contaminants are present.

The mixture method for the test sample and nucleic acid extraction reagent is not particularly limited, and the method suitably fit the state of test samples can be employed. For example, when the test sample is a suspension, mixture can be carried out by adding the nucleic acid extraction reagent to the test sample. Moreover, for example, when the test sample contains a large amount of colonies or solid materials, mixture can be carried out by suspending these materials in buffer solutions (e.g., PBS) or saline, which are commonly employed in biochemical experiments, and adding the nucleic acid extraction reagent to the suspensions.

Moreover, the mixture for the test sample and the nucleic acid extraction reagent can be carried out in a closable container. In such case, transportation of the test sample and the nucleic acid extraction reagent to the container can be carried out by using equipments such as pipettes or eye drop bottles.

The mixing ratio (volume ratio) of the test sample and the nucleic acid extraction reagent in the mixture is not particularly limited, for example, it can be in a range of 1:1000 to 1:10. Preferably, it can be in a range of 1:100 to 1:10.

Next, the mixture is modulated to a predetermined temperature, and left for a certain period.

As for the temperature of the mixture, for example, when a protease is contained in the nucleic acid extraction reagent, it can be a temperature close to the optimum temperature of the protease (e.g., in a range of 5° C.-lower to 5° C.-higher temperature than the optimal temperature).

Moreover, when protease is not contained in the nucleic acid extraction reagent, the temperature of the mixture can be in a range of 25° C. to 70° C.

Furthermore, the leaving time is not particularly limited; for example, it can be 5 to 30 minutes, and preferably, it can be 5 to 10 minutes.

Next, when protease is contained in the nucleic acid extraction reagent, the mixture is left for a certain period at a temperature close to the deactivation temperature of the protease (e.g., in a range of 2° C.-lower to 2° C.-higher temperature than the deactivation temperature).

The settling time is not particularly limited; for example, it can be 3 to 15 minutes, and preferably, it can be 3 to 5 minutes.

With the above operations, extracting solution with nucleic acids extracted from the test sample in the mixture is obtained.

The extracting solution obtained in such a way can be used for gene amplification reaction. When the objective nucleic acids for amplification are DNA, the obtained extracting solution is directly used as DNA templates in gene amplification reaction and is usable for PCR, and the supernatant after removing sediment comprising contaminants by operations of centrifugation or filtration also can be used for PCR.

When the objective nucleic acids for amplification are RNA, DNA in the mixture is further degraded, and the mixture after degrading treatment is used for RT-PCR.

In such case, as present in FIG. 1, this embodiment further comprises a process of contacting the DNA-degrading reagent (Reagent 2) with the extracting solution.

The above process can be carried out by adding the DNA-degrading reagent included in the nucleic acid extraction reagent kit to the extracting solution, modulating the extracting solution to a predetermined temperature, and leaving it for a certain period.

Such temperature, for example, can be a temperature close to the optimum temperature of the deoxyribonuclease contained in the DNA-degrading reagent (e.g., in a range of 2° C.-lower to 2° C.-higher temperature than the optimum temperature).

Moreover, the leaving period is not particularly limited if DNA is sufficiently degradable within the period. For example, it can be 10 to 30 minutes. Preferably, it can be 10 to 15 minutes.

Next, the extracting solution is left for a certain period at a temperature close to the deactivation temperature of the deoxyribonuclease (e.g., in a range of 2° C.-lower to 2° C.-higher temperature than the deactivation temperature).

The leaving period is not particularly limited; for example, it can be 5 to 30 minutes, and preferably, it can be 5 to 15 minutes.

According to the above operations, DNA can be removed and the extracting solution containing RNA can be obtained. RNA remained in the extracting solution after the above-mentioned treatment can be used as templates in RT-PCR method.

The present invention is explained in detail based on the above suitable embodiment.

However, the present invention is not limited to this. Each component can be displaced by anything that plays the same function, or any component can be added.

### EXAMPLES

The present invention is further explained in detail by providing examples as follows, but not limited to these examples.

Firstly, Reagent 1 and Reagent 2 with the following composition were prepared as reagents for extracting nucleic acids, and the nucleic acid extraction reagent kit comprising Reagent 1 and Reagent 2 was prepared.

Reagent 1 (Nucleic acid extraction reagent of the present invention)

| | | |
|---|---|---|
| Protease K | 18 | U/mL |
| Tris-HCl pH 7.5 | 0.9 | mM |
| Calcium acetate | 0.09 | mM |
| Sodium glycocholate | 9 | mM |
| EDTA•2Na | 0.9 | mM |
| Tricine pH 8.5 | 182 | mM |
| Sodium chloride | 9 | mM |
| Glycerin | 5% | (v/v) |

Reagent 2 (DNA-degrading reagent)

| | | |
|---|---|---|
| DNase I | 1 | U/μL |
| Tris-HCl pH 7.5 | 20 | mM |
| Magnesium chloride | 50 | mM |
| Glycerin | 50% | (v/v) |

In this connection, the compositions of Reagent 1 and Reagent 2 that are described below are those described above, unless otherwise stated.

Moreover, in the present examples, Reagent 1 and Reagent 2 were principally used as follows in the extraction of nucleic acids.

When the object for amplification was derived from DNA: the sample (1 μL) was suspended by Reagent 1 (100 μL), and then the suspension was continuously heated for 6 minutes at 65° C. and 3 minutes at 94° C. Then, the extracting solution was obtained by collecting the supernatant after the operation of centrifugation (10,000×g, 5 minutes), and used as the template for PCR.

When the object for amplification was derived from RNA: the same as the case of DNA, the sample (1 μL) and Reagent 1 (100 μL) were suspended, and the supernatant was collected through heating and centrifuging operations. 5 μL Reagent 2 and 45 μL ultrapure water were added to 5 μL of this supernatant. After continuous heating for 15 minutes at 37° C. and 5 minutes at 75° C., the extracting solution was obtained and used as the template for RT-PCR.

### Experimental Example 1

### DNA Extraction using Reagent 1 with Different Concentration of Protease K

Budding yeasts (Saccharomyces cerevisiae, ATCC 9763) were inoculated onto Sabouraud's dextrose agar medium and incubated aerobically overnight at 37° C. The obtained colonies were used as test samples. The above Reagent 1 was used with a stepwise change in concentration only for Protease K in a range of 0 to 45 U/mL. DNA as the object for amplification, nucleic acid extraction from test samples was carried out, and template for PCR was prepared.

Next, in order to obtain a 939-bp PCR product, sense primer (SEQ ID NO.1: URA3-U, 5′-GCACAGAACAAAAACCT-3′) and anti-sense primer (SEQ ID NO.2: URA3-L, 5′-TCATTACGACCGAGATT-3′) were used to amplify URA3 gene sequence by PCR. Firstly, 1 μL of forward primer (10 μM), 1 μL of reverse primer (10 μM), 4 μL of 5×APTATAQ® DNA Master (Roche), 2 μL of above-mentioned template and 120 μL of ultrapure water were mixed, and PCR reaction mixture with a whole volume of 20 μL was prepared. Then, PCR was carried out under the condition of temperature as follows: [(94° C., 30 sec; 48° C., 90 sec; 72° C., 60 sec)ₓ₃₀ cycles; 72° C., 7 min]. The amplified products were then subjected to electrophoresis and the result is shown in FIG. 2.

In the figure, the concentrations of Protease K in Reagent 1 for the experiments of each lane were comprised as follows.

Lane 1: 0 U/mL

Lane 2: 0.009 U/mL

Lane 3: 0.09 U/mL

Lane 4: 0.9 U/mL

Lane 5: 9 U/mL

Lane 6: 45 U/mL

Lane M: 100 bp DNA Ladder

As for the result shown by FIG. 2, it was confirmed that DNA was extracted and amplified for the rest samples performed by all these methods. Moreover, it was confirmed that the amount of amplified product was relatively large when the concentration of Protease K in Reagent 1 was in a range of 0.09 to 45 U/mL.

### Experimental Example 2

### DNA Extraction Using Reagent 1 with Different Concentration of Glycocholic Acid

Budding yeasts (Saccharomyces cerevisiae, ATCC 9763) were inoculated onto Sabouraud's dextrose agar medium and incubated aerobically overnight at 37° C. The obtained colonies were used as test samples. The above Reagent 1 was used with a stepwise change in concentration only for sodium glycocholate in a range of 0 to 9 mmol/L. DNA as the object for amplification, nucleic acid extraction from test samples was carried out, and template for PCR was prepared.

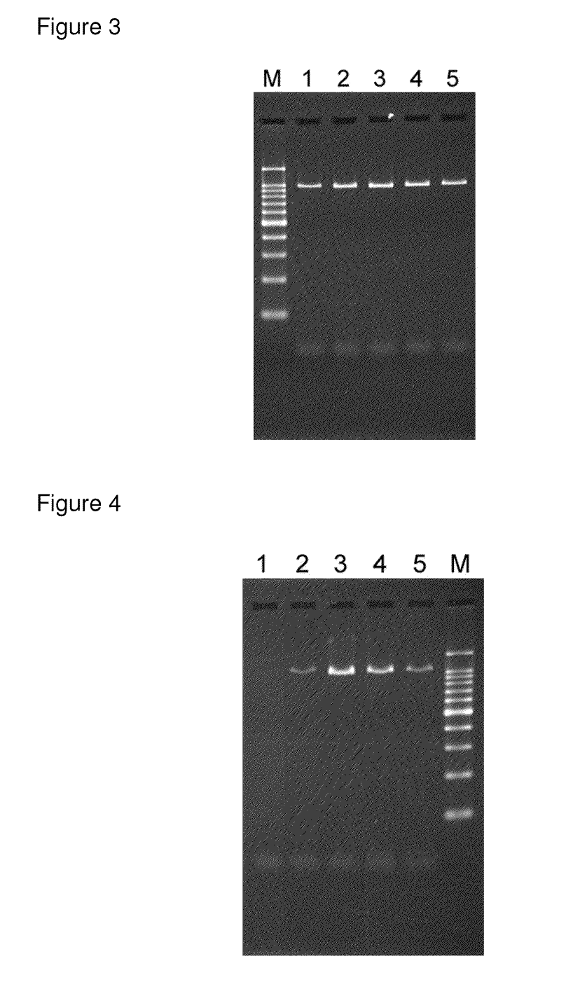

Next, in order to obtain a 939-bp PCR product, sense primer (SEQ ID NO.1: URA3-U, 5′-GCACAGAACAAAAACCT-3′) and anti-sense primer (SEQ ID NO.2: URA3-L, 5′-TCATTACGACCGAGATT-3′) were used to amplify URA3 gene sequence by PCR. Firstly, 1 μL of forward primer (10 μM), 1 μL of reverse primer (10 μM), 4 μL of SxAPTATAQ® DNA Master (Roche), 2 μL of the above-mentioned template and 12 μL of ultrapure water were mixed, and PCR reaction mixture with a whole volume of 20 μL was prepared. Then, PCR was carried out under the condition of temperature as follows: [(94° C., 30 sec; 48° C., 90 sec; 72° C., 60 sec)×30 cycles; 72° C., 7 min]. The amplified products were then subjected to electrophoresis and the result is shown in FIG. 3.

In the figure, the concentrations of sodium glycocholate in Reagent 1 for the experiments of each lane were comprised as follows.

Lane M: 100 bp DNA Ladder

Lane 1: 0 mmol/L

Lane 2: 0.009 mmol/L

Lane 3: 0.09 mmol/L

Lane 4: 0.9 mmol/L

Lane 5: 9 mmol/L

As for the result shown by FIG. 3, it was confirmed that the amount of amplified product was relatively large when the concentration of sodium glycocholate in Reagent 1 was in a range of 0.009 to 9 mmol/L.

### Experimental Example 3

### DNA Extraction Using Reagent 1 with Different Concentration of Tricine

Budding yeasts (Saccharomyces cerevisiae, ATCC 9763) were inoculated onto Sabouraud's dextrose agar medium and incubated aerobically overnight at 37° C. 1 μL of the obtained colonies were suspended in 10 μL of a human pooled serum, and the whole volume was used as test samples. The above Reagent 1 was used with a stepwise change in concentration only for tricine in a range of 0 to 364 mmol/L. DNA as the object for amplification, nucleic acid extraction from test samples was carried out, and template for PCR was prepared. Further, the Reagent 1 without tricine was used as a control.

Next, in order to obtain a 939-bp PCR product, sense primer (SEQ ID NO.1: URA3-U, 5′-GCACAGAACAAAAACCT-3′) and anti-sense primer (SEQ ID NO.2: URA3-L, 5′-TCATTACGACCGAGATT-3′) were used to amplify URA3 gene sequence.

Firstly, 1 μL of forward primer (10 μM), 1 μL of reverse primer (10 μM), 4 μL of 5×APTATAQ® DNA Master (Roche), 2 μL of template and 120 μL of ultrapure water were mixed, and PCR reaction mixture with a whole volume of 20 μL was prepared. Then, PCR was carried out under the condition of temperature as follows: [(94° C., 30 sec; 48° C., 90 sec; 72° C., 60 sec)ₓ₃₀ cycles; 72° C., 7 min]. The amplified products were then subjected to electrophoresis and the result is shown in FIG. 4.

In the figure, the concentrations of tricine in Reagent 1 for the experiments of each lane were comprised as follows.

Lane 1: 0 mmol/L

Lane 2: 9 mmol/L

Lane 3: 91 mmol/L

Lane 4: 182 mmol/L

Lane 5: 364 mmol/L

Lane M: 100 bp DNA Ladder

As for the result shown by FIG. 4, the bands derived from the amplified products were completely recognized when the concentration of tricine in Reagent 1 was in a range of 9 to 364 mmol/L.

### Experimental Example 4

### DNA Extraction from Staphylococcus Aureus (Gram-Positive Bacterium), Escherichia Coli O157 (Gram-Negative Bacterium) and Fungus (Budding Yeasts) using the Nucleic Acid Extraction Reagent Kit of the Present Invention

Staphylococcus aureus (NBRC 102141) and Escherichia coli O157 (ATCC 35150) were inoculated on to SCD agar medium and budding yeasts (Saccharomyces cerevisiae, ATCC 9763) were inoculated onto Sabouraud's dextrose agar medium respectively, and all of these were incubated aerobically overnight at 37° C. Each of the obtained colonies was then used as a test sample, and the above-mentioned Reagent 1 was used. DNA as the object for amplification, nucleic acid extraction from test samples was carried out, and template for PCR was prepared. Further, ultrapure water is used instead of the present invention (Reagent 1) and the same operation was carried out, which was used as a control template.

Next, for each template, 1 μL of forward primer (10 μM), 1 μL of reverse primer (10 μM), 4 μL of SxAPTATAQ® DNA Master (Roche), 2 μL of each template and 12 μL of ultrapure water were mixed, and each PCR reaction mixture with a whole volume of 20 μL was prepared, which were provided for DNA amplification reaction according to PCR reaction.

As for Staphylococcus aureus, in order to obtain a 324-bp PCR product, sense primer (SEQ ID NO.3: 54F, 5′-GACAACTAGAGATAGAGCCTTCC-3′) and anti-sense primer (SEQ ID NO.4: S4R,5′-AGTCGAGTTGCAGACTAC-3′) were used to amplify 16S rRNA sequence by PCR. PCR was carried out under the condition of temperature as follows: [(94° C., 30 sec; 54° C., 90 sec; 72° C., 60 sec)×30 cycles; 72° C., 7 min].

As for Escherichia coli O157, in order to obtain a 180-bp PCR product, sense primer (SEQ ID NO.5: stx1F, 5′-ATAAATCGCCATTCGTTGACTAC-3′) and anti-sense primer (SEQ ID NO.6: stx1R,5′-AGAACGCCCACTGAGATCATC-3′) were used to amplify Verocytotoxin (VT) gene sequence by PCR. PCR was carried out under the condition of temperature as follows: [(94° C., 30 sec; 62° C., 90 sec; 72° C., 60 sec)ₓ₃₀ cycles; 72° C., 7 min].

As for budding yeasts, in order to obtain a 939-bp PCR product, sense primer (SEQ ID NO:1: URA3-U, 5′-GCACAGAACAAAAACCT-3′) and anti-sense primer (SEQ ID NO:2: URA3-L, 5′-TCATTACGACCGAGATT-3′) were used to amplify URA3 gene sequence by PCR. PCR was carried out under the condition of temperature as follows: [(94° C., 30 sec; 48° C., 90 sec; 72° C., 60 sec)×30 cycles; 72° C., 7 min].

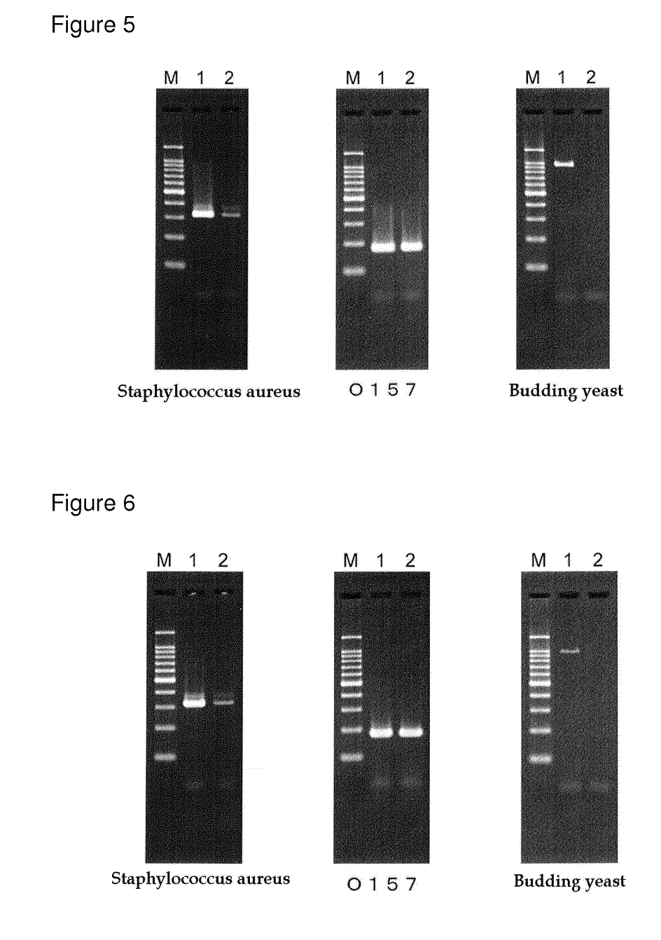

Each amplified product was then subjected to electrophoresis and the results are shown in FIG. 5.

In the figure, as for any of Staphylococcus aureus, enterohemorrhagic E. coli and budding yeast, each lane was composed as follows.

Lane M: 100 bp DNA Ladder

Lane 1: PCR template prepared using Reagent 1 of the present invention

Lane 2: PCR template prepared using ultrapure water instead of Reagent 1 of the present invention (Control)

As for the result shown by FIG. 5, it was confirmed that the bands derived from amplified products were detected for all of three test samples. Specifically, for Staphylococcus aureus and budding yeast, it was confirmed that more amplified products were obtained than the control.

### Comparative Example 1

### DNA Extraction from Staphylococcus Aureus (Gram-Positive Bacterium), Escherichia Coli O157 (Gram-Negative Bacterium) and Fungus (Budding Yeasts) using Conventional Nucleic Acid Extraction Reagent Kit

Instead of the nucleic acid extraction reagent kit of the present invention, an existing product (Cica Geneus DNA Extraction Reagent, manufactured by Kanto Chemical Co., Inc.) was used. Other than nucleic acid extraction and PCR template preparation, which were carried out according to the product manual of the same product, PCR template was prepared and amplified in the same way as the above-mentioned Experimental Example 4.

The amplified products were then subjected to electrophoresis and the results are shown in FIG. 6.

In the figure, as for any of Staphylococcus aureus, enterohemorrhagic E. coli and budding yeast, each lane was composed as follows.

Lane M: 100 bp DNA Ladder

Lane 1: Sample prepared using conventional nucleic acid extraction reagent kit (Cica Geneus DNA Extraction Reagent, manufactured by Kanto Chemical Co., Inc.)

Lane 2: Sample prepared using ultrapure water instead of the above reagent (Control)

As for the result shown by FIG. 6, for test samples of Staphylococcus aureus and O157, the bands that are derived from equal amplified products as the Experimental Example 4 were detected. However, it was confirmed that the amplified product was significantly less for the test sample of the budding yeasts.

### Experimental Example 5 and Reference Example 1

### DNA Extraction from Animal Cells using the Nucleic Acid Extraction Reagent Kit of the Present Invention or an Existing Product (CellEase Mouse Tail)

Mice's tails with a length about 3 mm were cut and used as animal test samples. Reagent 1 was used, DNA as the object for amplification, nucleic acid extraction from test samples was carried out, and template for PCR was prepared (Experimental Example 5). On the other hand, according to the existing product (CellEase Mouse Tail; Kanto Chemical), nucleic acid extraction from test samples was carried out and PCR template was prepared, which was used as a control (Reference Example 1).

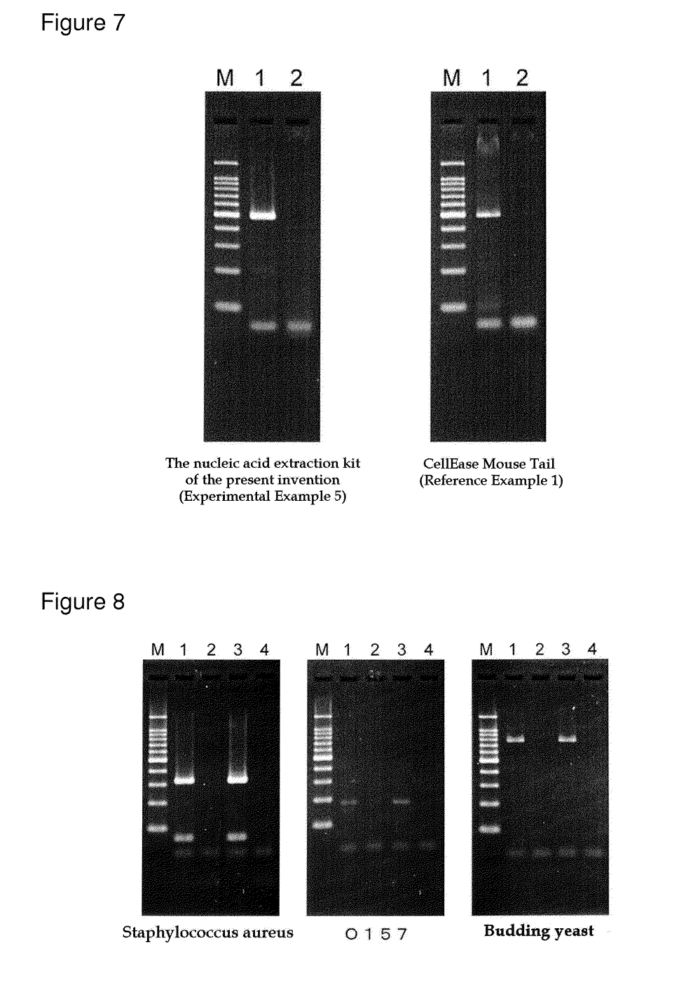

Next, in order to obtain a 494-bp PCR product, sense primer (SEQ ID NO.7: bGlo-F, 5′-CCAATCTGCTCACACAGGATAGAGAGGGCAGG-3′) and anti-sense primer (SEQ ID NO:8: bGlo-R, 5′-CCTTGAGGCTGTCCAAGTGATTCAGGCCATCG-3′) were used to amplify β-globin gene. Firstly, 0.4 μL of forward primer (10 μM), 0.4 μL of reverse primer (10 μM), 1.6 μL of MgCl₂ (25 mM), 1.6 μL of the mixture of each dNTP (2.5 mM), 2 μL of 10×Ex Taq Buffer (Mg²⁺ free, TAKARA BIO), 0.1 μL of TAKARA EX TAQ® (TAKARA BIO), 1.2 μL of 3-fold dilution of template in ultrapure water, and 12.7 μL of ultrapure water were mixed, and PCR reaction mixture with a whole volume of 20 μL was prepared. Then, PCR was carried out under the condition of temperature as follows: [94° C., 1 min; (94° C., 30 sec; 60° C., 30 sec; 72° C., 30 sec)ₓ₃₅ cycles; 72° C., 4 min]. The amplified products were then subjected to electrophoresis and the results are shown in FIG. 7.

In the figure, as for either of the nucleic acid extraction reagent kit of the present invention and the existing product (CellEase Mouse Tail; Kanto Chemical), each lane was composed as follows.

Lane M: 100 bp DNA Ladder

Lane 1: Sample prepared using Reagent 1 of the present invention or the existing product

Lane 2: Sample prepared using ultrapure water instead of Reagent 1 of the present invention or the existing product (Control)

As for the results shown by FIG. 7, it was confirmed that, when the nucleic acid extraction reagent kit of the present invention was used, the amount of amplified product was at least as large as that using the existing product, which specified objects to biological samples such as mouse's tail, beef and pork, etc.

### Experimental Example 6

### RNA Extraction from Staphylococcus Aureus (Gram-Positive Bacterium), Escherichia Coli O157 (Gram-Negative Bacterium) and Fungus (Budding Yeasts)

Staphylococcus aureus (NBRC 102141) and Escherichia coli O157 (ATCC 35150) were inoculated on to SCD agar medium and budding yeasts (Saccharomyces cerevisiae, ATCC 9763) were inoculated onto Sabouraud's dextrose agar medium respectively, and all of these were incubated aerobically overnight at 37° C. Each of the obtained colonies was then used as a test sample. The above-mentioned Reagent 1 and Reagent 2 were used. RNA as the object for amplification, nucleic acid extraction from test samples was carried out, and template for RT-PCR was prepared.

0.5 μL of reverse primer (2 μM, the same as that used for preparing PCR reaction mixture), 2 μL of SxPrimeScript Buffer for Real Time (TAKARA BIO), 0.5 μL of PrimeScript RT Enzyme Mix I (TAKARA BIO), 5 μL template and 2 μL ultrapure water were mixed, and the mixture for reverse transcription reaction with a whole volume of 10 μL was prepared. Then, the reverse transcription reaction was carried out under the condition of temperature as follows: [42° C., 15 min; 85° C., 5 sec]. Accordingly, the template after reverse transcription reaction was prepared.

Next, 1 μL of forward primer (10 μM), 1 μL of reverse primer (10 μM), 4 μL of SxAPTATAQ® DNA Master (Roche), 2 μL of the template after reverse transcription reaction and 12 μL ultrapure water were mixed, and the mixture for PCR reaction with a whole volume of 10 μL was prepared, which was provided for DNA amplification reaction according to PCR reaction. The condition of primers and temperature for each test sample is shown as follows

As for Staphylococcus aureus, in order to obtain 324-bp RT-PCR product, sense primer (SEQ ID NO.3: 54F, 5′-GACAACTAGAGATAGAGCCTTCC-3′) and anti-sense primer (SEQ ID NO.4: S4R,5′-AGTCGAGTTGCAGACTAC-3′) were used to amplify 16S rRNA sequence. PCR was carried out under the condition of temperature as follows: [(94° C., 30 sec; 54° C., 90 sec; 72° C., 60 sec)×30 cycles; 72° C., 7 min].

As for O157, in order to obtain a 180-bp RT-PCR product, sense primer (SEQ ID NO:5: stx1F, 5′-ATAAATCGCCATTCGTTGACTAC-3′) and anti-sense primer (SEQ ID NO:6: stx1R,5′-AGAACGCCCACTGAGATCATC-3′) were used to amplify Verocytotoxin (VT) gene sequence. PCR was carried out under the condition of temperature as follows: [(94° C., 30 sec; 62° C., 90 sec; 72° C., 60 sec)×30 cycles; 72° C., 7 min].

As for budding yeasts, in order to obtain a 756-bp RT-PCR product, sense primer (SEQ ID NO.9: ACT1f, 5′-TACGTTTCCATCCAAGCCGTT-3′) and anti-sense primer (SEQ ID NO.10: ACT1r, 5′-AACATACGCGCACAAAAGCAGA-3′) were used to amplify URA3 gene sequence. PCR was carried out under the condition of temperature as follows: [(94° C., 30 sec; 53° C., 90 sec; 72° C., 60 sec)×30 cycles; 72° C., 7 min].

The amplified products were then subjected to electrophoresis and the results are shown in FIG. 8.

In the figure, as for any of Staphylococcus aureus, enterohemorrhagic E. coli and budding yeast, each lane was composed as follows.

Lane M: 100 bp DNA Ladder

Lane 1: Sample prepared using Reagent 1 and Reagent 2 of the present invention (in a state with the addition of PrimeScript RT Enzyme Mix I)

Lane 2: Sample prepared using Reagent 1 and Reagent 2 of the present invention (in a state without the addition of PrimeScript RT Enzyme Mix I)

Lane 3: Sample prepared using ultrapure water (instead of Reagent 1 of the present invention) and Reagent 2 (in a state with the addition of PrimeScript RT Enzyme Mix I)

Lane 4: Sample prepared using ultrapure water (instead of Reagent 1 of the present invention) and Reagent 2 (in a state without the addition of PrimeScript RT Enzyme Mix I)

As for the results shown by FIG. 8, the bands derived from amplified products were confirmed for all of the three test samples.

### Experimental Example 7 and Comparative Example 2

### Nucleic Acid Extraction for Fungi (Budding Yeasts) Samples Contaminated with Blood Component

Budding yeasts (Saccharomyces cerevisiae, ATCC 9763) were inoculated onto Sabouraud's dextrose agar medium and incubated aerobically overnight at 37° C. 1 μL of the collected colonies were suspended in 10 μL of a human pooled serum, and the whole volume was used as a test sample.

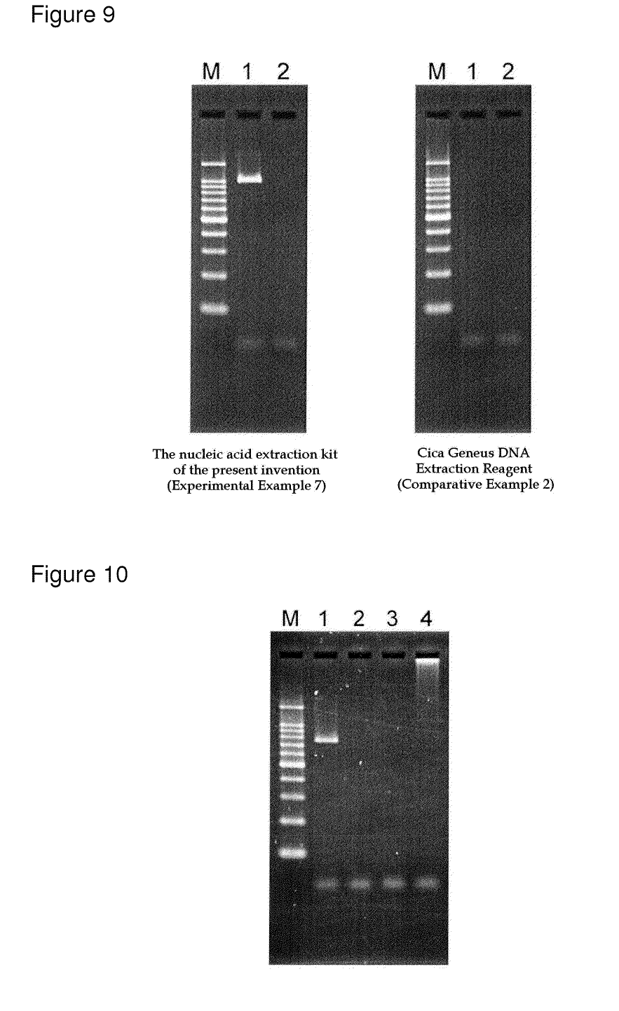

For this test sample, DNA extraction and PCR were carried out in the same way as Experimental Example 4, while RNA extraction and RT-PCR were carried out in the same way as Experimental Example 6, to obtain amplified products (Experimental Example 7). As a control, an existing product (Cica Geneus DNA Extraction Reagent; Kanto Chemical) was used instead of the nucleic acid extraction reagent kit of the present invention, and template for PCR was prepared in the same way as Comparative Example 1. Further, Preparation of RT-PCR template was not carried out, as the existing product is not applicable for RNA. The amplified products were then subjected to electrophoresis and the results are shown in FIG. 9 (PCR) and FIG. 10 (RT-PCR).

In FIG. 9, for either of the nucleic acid extraction reagent kit of the present invention and the existing product (Cica Geneus DNA Extraction Reagent; Kanto Chemical), each lane was composed as follows.

Lane M: 100 bp DNA Ladder

Lane 1: Sample prepared using Reagent 1 of the present invention or the existing product

Lane 2: Sample prepared using ultrapure water instead of Reagent 1 of the present invention or the existing product

Moreover, in FIG. 10, each lane was composed as follows.

Lane M: 100 bp DNA Ladder

Lane 1: Sample prepared using the nucleic acid extraction reagent kit (Reagent 1 and Reagent 2) of the present invention (in a state with the addition of PrimeScript RT Enzyme Mix I)

Lane 2: Sample prepared using the nucleic acid extraction reagent kit (Reagent 1 and Reagent 2) of the present invention (in a state without the addition of PrimeScript RT Enzyme Mix I)

Lane 3: Sample prepared using ultrapure water (instead of Reagent 1 of the present invention) and Reagent 2 (in a state with the addition of PrimeScript RT Enzyme Mix I)

Lane 4: Sample prepared using ultrapure water (instead of Reagent 1 of the present invention) and Reagent 2 (in a state without the addition of PrimeScript RT Enzyme Mix I)

As for the results shown by FIG. 9 and FIG. 10, when the nucleic acid extraction reagent kit of the present invention was used, DNA and RNA were detected even with the contamination of serum (blood component). However, in the control experiment using the existing product, DNA was not detected. Accordingly, it was confirmed that, no matter whether contaminants were present or not, nucleic acid extraction and nucleic acid sequence based amplification as the subsequent step could be performed according to the method of the present invention.

Accordingly, it was confirmed that, the nucleic acid extraction reagent kit and the nucleic acid extraction method of the present invention are applicable for various test samples, and nucleic acid templates directly usable for gene amplification reaction such as PCR and RT-PCR can be prepared conveniently and promptly, preferably, under a mild condition.

1. A nucleic acid extraction method comprising a process of contacting a nucleic acid extraction reagent with a test sample, wherein the nucleic acid extraction reagent comprises a zwitterionic buffer solution.

2. The nucleic acid extraction method according to claim 1, wherein the nucleic acid extraction reagent comprises at least one protease and/or at least one surfactant.

3. The nucleic acid extraction method according to claim 2, wherein the protease comprises Protease K.

4. The nucleic acid extraction method according to claim 2, wherein concentration of the protease in the nucleic acid extraction reagent is 0.09 to 45 U/mL.

5. The nucleic acid extraction method according to claim 2, wherein the surfactant comprises that having a steroid backbone.

6. The nucleic acid extraction method according to claim 2, wherein the surfactant comprises glycocholic acid or its salt.

7. The nucleic acid extraction method according to claim 2, wherein concentration of the surfactant in the nucleic acid extraction reagent is 0.009 to 9 mmol/L.

8. The nucleic acid extraction method according to claim 1, wherein the zwitterionic buffer solution is Good's buffer.

9. The nucleic acid extraction method according to claim 1, wherein the zwitterionic buffer solution comprises tricine.

10. The nucleic acid extraction method according to claim 1, wherein concentration of the buffer in the nucleic acid extraction reagent is 9 to 364 mmol/L.

11. The nucleic acid extraction method according to claim 1, wherein the test sample is a sample derived from organisms having cell walls.

12. The nucleic acid extraction method according to claim 11, wherein the organisms having cell walls comprises Gram-positive bacterium, fungus, or yeasts.

13. The nucleic acid extraction method according to claim 1, wherein DNA and/or RNA are/is extracted as nucleic acids from the test samples.

14. The nucleic acid extraction method according to claim 1, wherein the nucleic acid extraction reagent that is contacted with the test sample and extract nucleic acids from the test sample is used for nucleic acid amplification reaction.

15. A nucleic acid extraction reagent comprising a zwitterionic buffer solution.

16. A nucleic acid extraction reagent kit comprising a zwitterionic buffer solution and each component that compose a nucleic acid extraction reagent.


SEQUENCE LISTING (10 sequences)

SEQ ID NO: 1 - DNA, 17 bp, Artificial Sequence
  URA3-U
gcacagaacaaaaacct

SEQ ID NO: 2 - DNA, 17 bp, Artificial Sequence
  URA3-L
tcattacgaccgagatt

SEQ ID NO: 3 - DNA, 23 bp, Artificial Sequence
  S4F
gacaactagagatagagccttcc

SEQ ID NO: 4 - DNA, 18 bp, Artificial Sequence
  S4R
agtcgagttgcagactac

SEQ ID NO: 5 - DNA, 23 bp, Artificial Sequence
  stx1F
ataaatcgccattcgttgactac

SEQ ID NO: 6 - DNA, 21 bp, Artificial Sequence
  stx1R
agaacgcccactgagatcatc

SEQ ID NO: 7 - DNA, 32 bp, Artificial Sequence
  bGlo-F
ccaatctgctcacacaggatagagagggcagg

SEQ ID NO: 8 - DNA, 32 bp, Artificial Sequence
  bGlo-R
ccttgaggctgtccaagtgattcaggccatcg

SEQ ID NO: 9 - DNA, 21 bp, Artificial Sequence
  ACT1f
tacgtttccatccaagccgtt

SEQ ID NO: 10 - DNA, 22 bp, Artificial Sequence
  ACT1r
aacatacgcgcacaaaagcaga